(19) United States
(12) Patent Application Publication
(10) Pub. No.: US 2022/0323489 A1
(43) Pub. Date: Oct. 13, 2022

(54) HYPOCHLOROUS ACID SOLUTIONS FOR THE TREATMENT OF DRUG RESISTANT PNEUMONIA AND TUBERCULOSIS

(71) Applicant: Wonder Spray, LLC, San Diego, CA (US)

(72) Inventor: John Burd, San Diego, CA (US)

(73) Assignee: Wonder Spray, LLC, San Diego, CA (US)

(21) Appl. No.: 17/850,003

(22) Filed: Jun. 27, 2022

Related U.S. Application Data
(63) Continuation-in-part of application No. 17/518,452, filed on Nov. 3, 2021.
Application No. 17/518,452 is a continuation-in-part of application No. 17/179,251, filed on Feb. 18, 2021.
Application No. 17/179,251 is a continuation-in-part of application No. 16/833,209, filed on Mar. 27, 2020.
Application No. 16/833,209 is a continuation of application No. 16/293,551, filed on Mar. 5, 2019.

(51) Int. Cl. A61K 33/20; A61K 9/08; A61K 9/00; A61P 33/02; A61P 31/10; A61P 31/12; A61P 31/04

(52) U.S. Cl. CPC A61K 33/20; A61K 9/08; A61K 9/0078; A61P 33/02; A61P 31/10; A61P 31/12; A61P 31/04; A61K 9/006

(57) ABSTRACT

Systems and methods for treating drug resistant pneumonia and tuberculosis using hypochlorous acid (HOCl) are disclosed. Embodiments of the present disclosure relate to nebulizing HOCl as a method of treating drug resistant bacteria, in particular, bacterial pneumonia pathogens (i.e., Streptococcus pneumoniae) and Mycobacterium tuberculosis bacteria. The nebulized HOCl solution may be approximately 180 parts per million (PPM) of the aqueous solution and have a pH of less than 6.

### CROSS-REFERENCE TO RELATED APPLICATIONS

The present application is a continuation-in-part of U.S. patent application Ser. No. 17/518,452, filed on Nov. 3, 2021, which is a continuation in-part of U.S. patent application Ser. No. 17/179,251, filed Feb. 18, 2021, which is a continuation-in-part of U.S. patent application Ser. No. 16/833,209, filed on Mar. 27, 2020, which is a continuation of U.S. patent application Ser. No. 16/293,551, filed on Mar. 5, 2019, the contents of which are incorporated herein by reference in their entirety.

### TECHNICAL FIELD

The present disclosure is generally related to inhibiting activity and growth of pathogens and treating drug resistant pneumonia and tuberculosis with hypochlorous acid (HOCl). Embodiments of the present disclosure relate to nebulizing HOCl as a method of treating drug resistant bacteria, in particular, bacterial pneumonia pathogens (i.e., Streptococcus pneumoniae) and Mycobacterium tuberculosis bacteria.

### BACKGROUND

Antimicrobial medications are commonly used to treat infectious disease. Antimicrobial resistance is a key issue that needs to be taken into account when selecting a therapeutic agent for the treatment of infectious diseases. For example, infections due to multidrug-resistant bacteria, such as multidrug-resistant tuberculosis (MDR-TB) or drug resistant pneumonia, are serious threats. Although bacterial resistance is a natural phenomenon, the misuse of antimicrobial drugs has accelerated the development of resistance to infectious diseases. Accordingly, a treatment that an infectious agent cannot develop a resistance to would be advantageous.

Pneumonia is a serious infection caused by bacterial or viral infections in the lungs, in which air sacs fill with pus and may become solid. Pneumonia can affect one (single pneumonia) or both lungs (double pneumonia) of patients, and may affect single (lobar pneumonia) or multiple lobes within each lung. Similarly, tuberculosis (TB) is a serious infectious disease that primarily affects the lungs of patients. TB is caused by Mycobacterium tuberculosis bacteria that can spread from person to person through droplets released into the air via coughing and sneezing. Although antibiotics and antimicrobial medications have conventionally been used to treat pneumonia and TB, drug resistant strains of pneumonia and TB have begun to emerge. Accordingly, a treatment for drug resistant pneumonia and MDR-TB that cannot have a resistance developed against it would be advantageous.

### SUMMARY

Embodiments of the present disclosure may be directed to systems and methods for prevention and treatment of infectious diseases of the lung using low concentration hypochlorous acid solutions. Some embodiments may relate to prevention and treatment of drug resistant pneumonia and tuberculosis using low concentration hypochlorous acid solutions. One aspect of the present disclosure relates to a method of delivering to a patient a hypochlorous acid solution via a pulmonary delivery apparatus.

Another aspect of the present disclosure relates to a method of destroying infectious agents and organisms on medical equipment used by a patient to prevent infection by the infectious diseases in the patient. The method may include delivering a hypochlorous acid solution to the medical equipment to disinfect the medical equipment used by the patient, thereby preventing infection by the infectious diseases in the patient. The hypochlorous acid solution may include an aqueous solution of hypochlorous acid. The hypochlorous acid may be approximately 180 parts per million (PPM) of the aqueous solution.

In embodiments, the hypochlorous acid may have a pH range of less than 6.

In embodiments, the delivery of the solution may include placing the aqueous solution of the hypochlorous acid within a reservoir of a pulmonary delivery apparatus. The delivery of the solution may include transforming the aqueous solution of the hypochlorous acid into a mist. The delivery of the solution may include spraying the mist of the aqueous solution of hypochlorous acid into the mouth or nose of a patient.

In embodiments, the pulmonary delivery apparatus may include CPAP equipment, BiPAP equipment, asthma nebulizers/nebulizers, oxygen concentrators, medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, asthma nebulizer, and oxygen concentrators.

### BRIEF DESCRIPTION OF THE DRAWINGS

FIG. 1 illustrates a perspective view of an example pulmonary delivery apparatus for administering HOCl comprising a nebulizer, according to an implementation of the disclosure.

FIG. 2 illustrates a process of administering HOCl using a pulmonary delivery apparatus, such as a nebulizer, according to an implementation of the disclosure.

FIG. 3 illustrates tables showing an example effect of a solution of HOCl on Streptococcus pneumoniae, according to an implementation of the disclosure.

FIG. 4 illustrates tables showing an example effect of a solution of HOCl on Streptococcus pyogenes, according to an implementation of the disclosure.

### DETAILED DESCRIPTION

Hypochlorous acid (HOCl) is a weak acid that forms when sodium chloride dissolves in water, and itself partially dissociates, forming HOCL and hypochlorite, OCl−, depending on the solution pH. Similar to other chlorine-releasing agents (e.g., sodium hypochlorite, chlorine dioxide, and the N-chloro compounds such as sodium dichloroisocyanurate), aqueous HOCL is well known for its antimicrobial, anti-inflammatory, and immunomodulatory properties.

Applications of aqueous solutions containing approximately 30-2500 ppm (0.003% to 0.25%) HOCl are used in a variety of areas including (but not limited to) wound care, as antimicrobial agents, as anti-allergen agents, dental care and there are also significant applications in disease treatments, water treatments, food sanitization, hard surface disinfection, and cosmetics. HOCl can also be used as a disinfectant in medical equipment, including CPAP equipment, BiPAP equipment, asthma nebulizers/nebulizers, oxygen concentrators, medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, and/or other medical equipment. HOCl can also be used therapeutically and/or prophylactically to prevent or limit the spread of infectious organisms, clear the natural flora of a known contaminating pathogen, and/or to sterilize spaces contaminated with hard to eradicate pathogens.

HOCl is a potent antimicrobial capable of eradicating bacteria including antibiotic-resistant strains, such as multidrug resistant tuberculosis and drug resistant pneumonia, viruses, yeast, fungi, and spores. In particular, HOCl is the active component responsible for pathogen disruption and inactivation by chlorine-releasing agents (CRAB). It is understood that the OCl− ion has little effect compared to undissolved HOCl, as the hypochlorite (OCl−), has only a minute effect compared to undissolved HOCl. Accordingly, the microbicidal effect of HOCl is the greatest when the percentage of undissolved HOCl is highest. For example, one embodiment of the present disclosure may include delivery an aqueous solution of HOCl to a patient, where the aqueous solution has a concentration of 180 PPM of HOCl and a pH of less than 6. In an aqueous solution of HOCl, ranging from approximately pH 4 to pH 7, chlorine exists predominantly as HOCl, whereas above pH 7.5, ClO− predominates.

Because HOCl is a highly active oxidizing agent, its mode of operation comprises destroying and/or deactivating cellular activity of proteins. For example, HOCl targets bacteria by chemically linking chlorine atoms to nucleotide bases that disrupt the function of bacterial DNA, impede metabolic pathways in which cells use enzymes to oxidize nutrients, and release energy, and other membrane-associated activities. Additionally, HOCl has also been found to disrupt oxidative phosphorylation and other membrane-associated activity. Similarly, HOCl has been found to inhibit bacterial growth. For example, at 50 mM (2.6 ppm), HOCl completely inhibited the growth of E. coli within 5 minutes, including inhibiting the DNA synthesis by ninety-six percent. Unlike conventional antibiotics, the antimicrobial activity of HOCl is directly toxic to microbial cells, including many Gram-positive and Gram-negative bacteria and their biofilms. HOCl has demonstrated disinfection efficacy against eradication of bacteria, including Acinetobacter baumannii, Bacillus subtilis, Enterobacter cloacae, Enterococcus faecalis, Escherichia coli, Enterobacter, Klebsiella pneumoniae, Listeria monocytogenes, MRSA (Staph. aureus), Mycobacterium Tuberculosis, Polymicrobial biofilm, Proteus vulgaris, Pseudomonas aeruginosa, Salmonella choleraesuis, Shigella flexneri, Staph epidermidis, and Yersinia enterocolitica. HOCl can also be used to treat, kill, disinfect, minimize levels of, and/or otherwise affect infectious disease agents, infectious diseases, and/or complications after infections, including mast cell degranulation, acne, tuberculosis, pneumonia, Streptococcus pyogenes, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, Staphylococcus aureus, Haemophilius influenzae, Klebsiella pnuemoniae, Pseudomona aeruginosa, Bordetella pertussis, Moraxella catarrhalis, Coxiella burnetiid, Chlamdyophilia pneumoniae, Mycoplasma pneumoniae, Legionella pneumophilia, Yesinia pestis, influenza viruses, rhinoviruses, respiratory syncytial virus, adenovirus, enterovirus, parainfluenza, Epstein-Barr virus, cytomegalovirus, hantavirus, Herpes simplex, Histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, SARS-CoV-2, and the like. HOCl can also be used as a prophylaxis for splenectomized patients and others at risk for pneumonia, which is a commonly found risk for patients who regularly use PAP equipment.

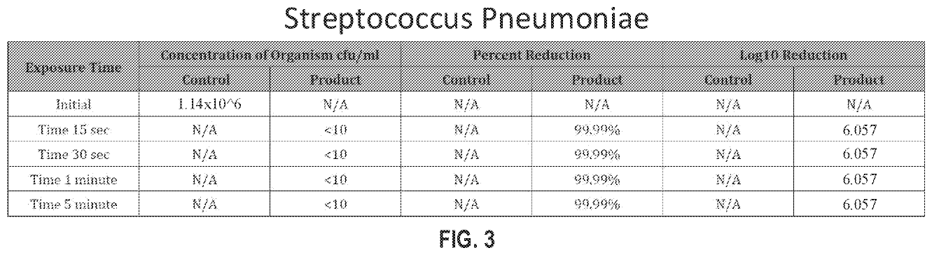

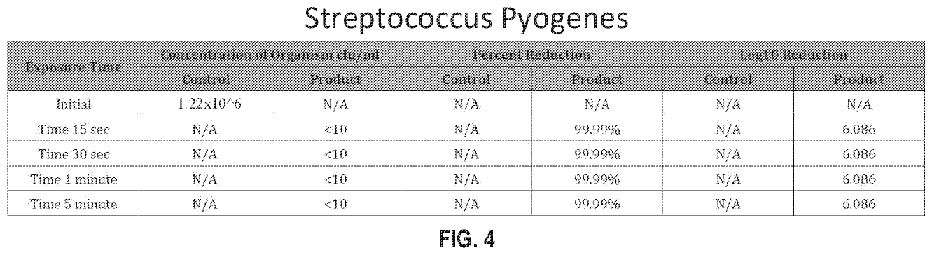

For example, FIGS. 3 and 4 illustrate tables of the effect of a solution of HOCl on Streptococcus pneumoniae and Streptococcus pyogenes, respectively. The organisms in this test were prepared by inoculating the surface of about 5% Sheep blood agar plates, incubated at about 30 to 35° C. for 18 to 24 hours. Following the incubation period, the plates were washed with sterile Serological Saline Solution to harvest the microorganisms used and dilutions with Saline were made, plated on blood agar and incubated at 30 to 35° C. for 24-48 hours to determine the concentration. The inoculum level was then adjusted to 108 CFU/ml for use as a stock suspension. Stock suspensions were well mixed and homogenized at each inoculation interval. The following microorganisms were used in this Kill Time Study to demonstrate the antimicrobial properties of the HOCl solution against common pathogenic organisms: Microbiologies Kwik-Stik Streptococcus pyogenes ATCC 49399, Streptococcus pneumoniae ATCC 49619.

Positive controls were performed at initiation and completion by spread plating to enumerate inoculum levels and verify culture purity during testing and Negative controls were performed to establish sterility of media, reagents, and materials used at initiation. Neutralizer Suitability using Modified Letheen Broth (MLB) was performed concurrently with Kill Time testing to confirm the recovery of <100 CFU of the test organism in the subculture media in the presence of product. Duplicate 10 ml containers for each treated specimen or material concentration was prepared, equilibrated to 25±2° C., and 0.1 ml of inoculum is added to each container to achieve a final concentration of 106 CFU/ml.

Serial dilutions from each replicate were made at intervals of 15 second, 30 second, 1 minute and 5 minutes using 1 ml of the inoculated test product into 9 ml MLB from 1:10 to 1:1000000. Subsequently, 1 ml from each dilution was spread plated on 5% Sheep Blood agar plate in duplicate, incubated at 30 to 35° C. for 48 hours. After the incubation period, all plates were counted to determine the number of microorganisms, results are averaged and reported as log 10 reductions.

Referring back to FIG. 3, after fifteen seconds, there were less than 10 CFU/ml of the Streptococcus pneumoniae after the HOCl solution was applied, the percent reduction for Streptococcus pneumoniae was 99.99% after the HOCl solution was applied, and the log 10 reduction for Streptococcus pneumoniae was 6.057 after the HOCl solution was applied.

In FIG. 4, after fifteen seconds, there were less than 10 CFU/ml of the Streptococcus pyogenes after the HOCl solution was applied, the percent reduction for Streptococcus pyogenes was 99.99% after the HOCl solution was applied, and the log 10 reduction for Streptococcus pyogenes was 6.086 after the HOCl solution was applied.

FIGS. 3 and 4 illustrate that the bacteria that cause strep throat and pneumonia are effectively killed by HOCL in less than 15 seconds. The currently disclosed solution of HOCl overcomes the issues with other medicine commonly prescribed for these diseases, and is an effective treatment against these diseases, as well as other drug resistant infections. For example, healthcare professionals may prescribe penicillin or amoxicillin to treat these bacterial infections. Side effects of penicillin antibiotics include diarrhea, dizziness, heartburn, insomnia, nausea, itching, vomiting, confusion, abdominal pain, easy bruising, bleeding, rash, and allergic reactions. The most common side effects of amoxicillin are nausea, vomiting, stomach pain and diarrhea. Moreover, amoxicillin may also destroy the good bacteria that naturally resides in the body. This can lead to an overgrowth of yeast, which may not only produce diarrhea but also cause yeast infections, especially in the mouth and vagina. In addition, oral amoxicillin exposure may cause shifts in microbiome composition that can last approximately 30 days on average and more than 2 months in some individuals. This shift in microbiome composition can also predispose patients to Clostridium difficile colitis. The additional concern about resistance to antibiotics is well known. Bacteria have not been known to develop a resistance to the HOCL nor is it known to cause any of the side effects listed above.

Additionally, HOCl possesses viricidal activity properties. For example, it has been demonstrated that HOCl inactivated naked f2 RNA at the same rate as RNA in intact phage, whereas f2 capsid proteins could still adsorb to the host. HOCl has demonstrated disinfection efficacy against eradication of viruses including norovirus, filoviruses such as Ebola, and human coronaviruses like MERS-CoV, SARS, and SARS-CoV-2, as well as fungi such as Candida and Aspergillus. Further, as a sporicide, HOCl causes the spore coat to detach from the cortex, where further degradation occurs.

Both topical and internal applications of HOCl are safe because it is the exact same substance white blood cells in the human body produce to fight infection. Indeed, extensive studies have demonstrated exceptional safety of HOCl. The Food and Drug Administration (FDA) has cleared preparations of HOCl to be used, e.g., topically for wound cleansing, eye infections, tooth infections, nasal decontamination, and the care of surgical incisions. In particular, inhaling the aerosolized form of HOCl has also been shown to causes no adverse effects. As used, for example, in treating pneumonia and tuberculosis via a pulmonary delivery apparatus.

The advent of antibiotics and other area disinfectants led to a reduction in environmental use of HOCl. However, widespread use of antibiotic agents led to antimicrobial resistance. For example, MDR-TB was nonexistent before 1956, which was when the first cases began appearing in Great Britain. Accordingly, an urgent need to optimize currently available anti-infectious therapies to overcome drug resistance exists. Antimicrobial resistance has not been observed for HOCl.

Various embodiments of the systems and methods disclosed herein are directed to antimicrobial and antibiotic agent delivery systems, using various pulmonary delivery apparatuses, in which the antimicrobial and antibiotic agent is administered via a pulmonary route as a treatment for infectious diseases caused by microbes (e.g., bacteria, spores), viral, fungal, allergy-causing agents. In embodiments, the antimicrobial agent may be an aqueous solution of HOCl, which may be delivered to a patient via pulmonary delivery apparatus, such as a nebulizer, at approximately 180 PPM, with a pH of less than 6. One skilled in the art would appreciate that other concentrations and other pHs may provide similar therapeutic results. Because the inhalation process gives a more direct access to the target organ/cavity (i.e., lungs) than more traditional routes (e.g., topical, oral, intravenous, etc.), the pulmonary administration of HOCl used to inhibit bacterial growth provides a therapeutic approach that may help avoid and reduce antimicrobial resistance while alleviating and treating diseases, such as tuberculosis or pneumonia. For example, upper respiratory tract infections caused by one or more bacterial or viral pathogens such as tuberculosis, bronchitis, epiglottitis, laryngitis, sinusitis, rhinosinusitis, chronic rhinosinusitis or similar; and lung infections, such as pneumonia and tuberculosis, may be treated by a pulmonary administration of HOCl via a pulmonary delivery apparatus.

In some embodiments, a solution of HOCl may be delivered via the pulmonary route utilizing a number of pulmonary delivery apparatuses. For example, the pulmonary delivery apparatus may be a nebulizer, CPAP device, BiPAP device, oxygen concentrator, aerosolizer, atomizer, and/or any other such apparatus that can deliver an aqueous solution from outside the body of a patient to the lungs or pulmonary system of a patient. For example, a solution of HOCl of low concentration levels and relatively low acidotic pH, i.e., 180 PPM and <6 pH, may be used as a nebulized tuberculosis treatment. In some embodiments, the aqueous solution of HOCl may include a concentration of approximately 0.02 percent of HOCl dissolved in water.

In some embodiments, the HOCl solution may be diffused. The diffusion may be the result of nebulization, ultrasonication, and/or other mechanisms accomplished through pulmonary delivery apparatuses. Pulmonary delivery apparatuses may send air through a nebulizer device, which is then inhaled by a patient. The inhalation by the patient may allow for the nebulized HOCl to be administered to the lungs allowing for treatment of symptoms of pneumonia and/or tuberculosis and reduction of pneumonia and/or tuberculosis bacteria in the patient.

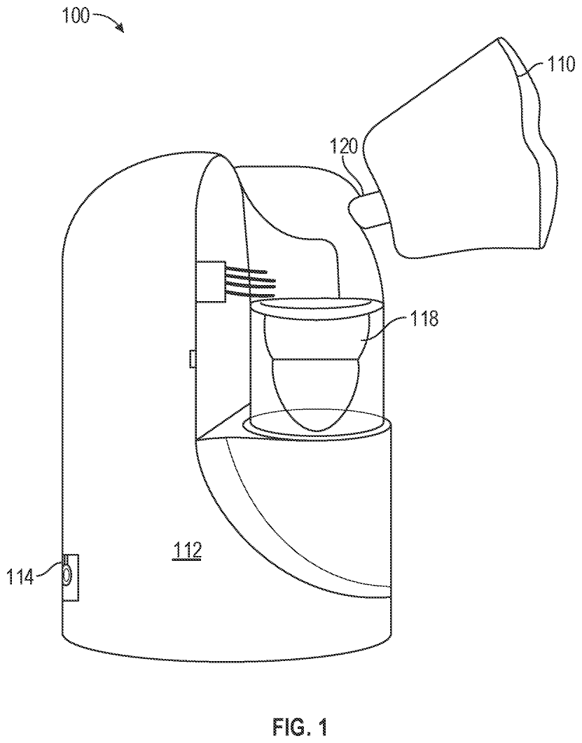

FIG. 1 illustrates a perspective view of an example pulmonary delivery apparatus for administering HOCl comprising a nebulizer, according to an implementation of the disclosure. The pulmonary delivery apparatus 100 or components/features thereof may be implemented in combination with, or as an alternative to, other systems/features/components described herein, such as those described with reference to other embodiments and figures. The pulmonary delivery apparatus 100 may additionally be utilized in any of the methods for using such systems/components/features described herein. The pulmonary delivery apparatus 100 may also be used in various applications and/or permutations, which may or may not be noted in the illustrative embodiments described herein. For instance, pulmonary delivery apparatus 100 may include more or less features/components than those shown in FIG. 1, in some embodiments. Moreover, the pulmonary delivery apparatus 100 is not limited to the size, shape, number of components, etc. specifically shown in FIG. 1.

As shown in FIG. 1, the pulmonary delivery apparatus 100 may comprise a housing 112 which may house one or more components configured to aerosolize the aqueous solution so that it can be administered it in the form of aerosolized particles by being inhaled into a patient's lungs. For example, the one or more components hosed in housing 112 may include an ultrasonic generator or oscillator, a compressor, or similar components and associated circuitry (not shown) for causing aerosolization.

Further, the pulmonary delivery apparatus 100 may comprise a liquid supply reservoir 118 and a mouthpiece 110. In some embodiments, the aqueous solution may be placed within the liquid supply reservoir 118. For example, the aqueous solution may include liquid HOCl liquid solution ranging from 0.5 ml to 20 ml placed in the reservoir 118.

In some embodiments, the one or components housed within housing 112 may cause the aqueous solution to aerosolize the aqueous solution. For example, the housing 112 may include an inlet (not shown) through which air is supplied under pressure from a compressor (not shown). In some embodiments, pulmonary delivery apparatus 100 may be configured to use a driving gas flow (typically 0.5 mL/min) to generate aerosol. For example, the nebulizers may deliver an approximately equal volume of aerosol during the inhalation phase (i.e., when patient is breathing). In other embodiments, an oscillator (not shown) may transmit ultrasonic waves through the aqueous solution.

In some embodiments, the pressurized air may be directed via an air channel (not shown) into the liquid supply reservoir 118 causing a rapid formation and collapse of bubbles, which then stream toward the surface of the solution and encounter the interface between the solution and air, resulting in a production of a fine mist or aerosol adjacent the solution surface.

In some embodiments, the pulmonary delivery apparatus 100 may be configured to carry the aerosol upwardly through a conduit 120 connected to the mouthpiece 110. The patient may aspirate the aerosolized aqueous solution through the mouthpiece 110. In some embodiments, the diameter of aerosol particles or droplets may be approximately 1 to 5 microns to ensure the particles or droplets are not likely to be impacted in the airway before they reach the lungs and are not carried out of the lungs again on exhalation without being deposited within the respiratory system structures (e.g., lungs).

In some embodiments, the conduit 120 may be configured to be slightly larger in diameter than an exit port (not shown) within the mouthpiece 110. By virtue of the conduit 120 being slightly larger in diameter than the air exit port of the mouthpiece 100 a small space between the outer surface of the air exit port and the inner surface of the conduit 120 is provided. For example, the space may be approximately 0.00254-0.254 mm. In some embodiments, the space allows fluid from the liquid supply reservoir 118 to proceed upward between the air exit port and the conduit 120. In some embodiments, the diameter of the conduit 120 may be adjusted to change the particle size of the mist.

In some embodiments, the housing 112 may include one or more pressure sensors (not shown) configured to detect the pressure within the liquid supply reservoir 118. In some embodiments, the one or more pressure sensors may be connected to the inside of the mouthpiece 100. In some embodiments, the one or more pressure sensors may detect that a patient has inhaled causing the pulmonary delivery apparatus 100 to divert pressurized air to an air outlet (not shown).

In some embodiments, the pulmonary delivery apparatus 100 may be configured to analyze the pressure changes within the apparatus 100 during a certain number of initial breaths (e.g., first three breaths) to determine an average shape of the breathing pattern. A timed pulse of atomization is commenced upon start of subsequent inspirations such that atomization occurs for the first 50 percent of the inspiration. In some embodiments, the pulmonary delivery apparatus 100 may be configured to have a timed pulse of atomization to occur during a period other than 50% of the duration of inspiration. In some embodiments, the pulmonary delivery apparatus 100 may be configured to have a predetermined pulse length. For example, the pulse length may be set for each patient by a clinician.

In some embodiments, the housing 112 may include one or more panels (not shown) to operate the one or more components configured to aerosolize the aqueous solution. In some embodiments, the housing 112 may comprise a connector 114 to which a power cable (not shown) may be connected.

In some embodiments, the nebulized HOCl may be delivered using compressor-based jet-nebulizer system. For example, a jet nebulizer may be configured with a mechanism to allow the production of aerosol HOCl only when the inhalation airflow exceeds a certain flow rate. By virtue of including the mechanism, the jet nebulizer provides control over the portion of the breath into which the aerosol HOCl is delivered.

In some embodiments, the nebulized HOCl may be delivered using a mesh-based nebulizer system. In some embodiments, the mesh-based nebulizer may be used to deliver aerosol HOCl based on a breathing-pattern of a patient. For example, the mesh-based nebulizer may include one or more sensors configured to monitor inspiratory flow rate and length of the inhalation. In some embodiments, aerosol HOCl may be pulsed during the first fifty to eighty percent of the inhalation, based on determined specific characteristics of the breathing pattern. The duration of each pulse of aerosol HOCl may be determined by the patient's breathing pattern and varied for each subsequent breath, depending on the preceding breaths.

In some embodiments, the nebulized HOCl may be delivered using an ultrasonic wave nebulizer. For example, the ultrasound wave nebulizer may comprise an electronic oscillator and a one or more piezoelectric elements to create an aerosol. The electronic oscillator may be configured to generate a high frequency ultrasonic wave, which causes the mechanical vibration of the one or more piezoelectric elements, the one or more piezoelectric elements may be in contact with a compartment used to store an aqueous HOCl solution. The one or more piezoelectric elements may vibrate at a high frequency and deliver a vapor mist comprising an aerosolized HOCl.

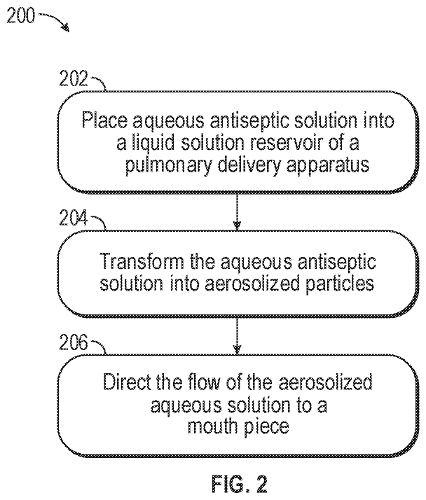

In some embodiments, illness suspected to be caused by microbes, bacteria (i.e., pneumonia and/or tuberculosis), spores, viral, fungal, allergy-causing agents may be treated by administering HOCl through use of aerosolized or atomized pulmonary delivery apparatus. Embodiments using this method involve the use of an aerosolizer or an atomizer to aerosolize a liquid HOCl solution for respirable delivery. For example, and as illustrated in FIG. 2, the delivery of the aerosolized HOCl may include one or more of the following operations. In an operation 202, an aqueous solution may be placed in a reservoir of an aerosolizing, atomizing, diffusing, or similar pulmonary delivery apparatus. For example, the aqueous solution comprising HOCl ranging in volume from approximately 0.5 ml to 10 ml may be placed into the reservoir. In an operation 204, the aqueous solution may be aerosolized into particle sizes. For example, the aqueous solution comprising HOCl may be aerosolized into particles ranging from approximately 0.1 μm to 99 μm or larger, in size. In an operation 206, the aqueous solution may be directed into a mouthpiece affixed to the device to be aspired by a patient into their upper respiratory track. For example, the aerosolized aqueous solution comprising HOCl may be aspired for a prescribed duration (e.g., a period ranging from approximately 0.5 minutes to 30 minutes or longer, with repetitions between 5 and 10 times per day).

In some embodiments, the solution may be diffused. The diffusion may be the result of nebulization, ultrasonication, and/or other mechanisms. A nebulizing diffuser may send air through a small tube at a high speed to produce pressure differentials in and around the tube (i.e., the pressure near the end of the tube is lower than in the tube). This pressure difference may cause a suction-like effect causing the solution to be sucked up toward the top of the tube where they are atomized into tiny particles in a mist.

An ultrasonic diffuser may use ultrasonic vibrations to convert the HOCl solution into a vapor. In some embodiments, this may be accomplished using a vibrating membrane.

In some embodiments, illness suspected to be caused by microbes, bacteria (i.e., pneumonia and/or tuberculosis), spores, viral, fungal, and/or allergy-causing agents may be treated by administering HOCl through use of aerosolized or atomized administration via patient's nasal sinus passages. For example, the delivery of the aerosolized HOCl may include one or more of the following: placing liquid solution ranging from 0.5 ml to 10 ml placed in the reservoir of an aerosolizing, atomizing, or similar device, aerosolizing or atomizing the liquid into particle sizes ranging from approximately 0.1 μm to 99 μm or larger, and inserting a nose piece affixed to the device into the nostril(s) for a prescribed duration (e.g., a period ranging from approximately 0.5 minutes to 10 minutes, or longer with repetitions between 5 and 10 times per day).

In some embodiments, illness suspected to be caused by microbes, bacteria, spores, viral, fungal, and/or allergy-causing agents may be treated by administering HOCl through use of a nasal spray. For example, a spray device comprising a pump and a spray nozzle, may be used to transform the aqueous solution of HOCl into a mist for administering the solution to the nasal passages and sinus cavities. In some embodiments, the delivery of the mist comprising an aqueous HOCl solution may include one or more of the following: placing an aqueous solution of HOCl ranging from 0.1 ml to 10 ml into a nasal spray bottle adequately suited for nasal use, and administering the mist comprising aqueous solution HOCl into the nasal passages of each nostril via the spray bottle by compressing the spray bottle. For example, approximately one to ten sprays per nostril may be administered into each nostril by compressing the spray bottle. In some embodiments, the aqueous solution of HOCl may be diluted with one or more diluents. For example, 0.5 ml to 20 ml of saline may be added to the aqueous solution of HOCl.

In some embodiments, illness suspected to be caused by microbes, bacteria (i.e., pneumonia and/or tuberculosis), spores, viral, fungal, and/or allergy-causing agents may be treated by administering HOCl through use of nasal rinsing or irrigation. Generally, nasal rinses and irrigation systems are used to flush out excess mucus and debris from the nasal passages and sinus cavities, but they can also be used to administer medicated solutions to the nasal passages and sinus cavities. In some embodiments, the delivery of the aqueous solution of HOCl may include one or more of the following: adding an aqueous solution of HOCl (ranging approximately from approximately 0.1 ml to 10 ml) to a commercially available or prepared nasal rinsing or irrigation solution, and administering the aqueous solution of HOCl to the nasal passages and sinus cavities by rinsing, flushing, irrigating, or otherwise exposing nasal passages and cavities to the combined aqueous solution of HOCl and rinsing or irrigating solution.

In some embodiments, illness suspected to be caused by microbes, bacteria (i.e., pneumonia or tuberculosis), spores, viral, fungal, and/or allergy-causing agents may be treated by administering HOCl through use of an oral rinse solution. Generally, oral rinses use a liquid solution that is used to swish around the oral cavity, including teeth, gums and tongue to help prevent or treat various oral health conditions and diseases (e.g., gum disease, canker sores, halitosis, gingivitis, tartar, and so on). In some embodiments, the aqueous solution of HOCl may be diluted with one or more diluents. For example, approximately 0.5 ml to 20 ml of saline may be added to the aqueous solution of HOCl. In some embodiments, a particular amount of aqueous solution of HOCl (e.g., approximately 5 mL) may be placed inside a patient's oral cavity without swallowing. While keeping the lips closed, the patient may make a swishing motion to move the HOCl so that so that the HOCl solution reaches the front and sides of your mouth equally for a prescribed duration (e.g., a period ranging from approximately 0.5 min to 10 min, or longer), repeating it as necessary. Upon completing the oral rinse, the HOCl solution may be spit out.

In some embodiments, illness suspected to be caused by microbes, bacteria (i.e., pneumonia and/or tuberculosis), spores, viral, fungal, and/or allergy-causing agents may be treated by administering HOCl through use of a gargling solution. For example, a particular amount of aqueous solution of HOCl (e.g., approximately 5 mL) may be placed inside a patient's oral cavity without swallowing. While keeping the lips and teeth slightly apart and tilting the head slightly backwards, the patient may move the liquid within the throat cavity by exhaling through it for a prescribed duration (e.g., a period ranging from approximately 0.5 min to 10 min, or longer), repeating it as necessary. Upon completing the gargling, the HOCl solution may be spit out. In some embodiments a spray device comprising a spray nozzle, may be used to transform the aqueous solution of HOCl into a mist for administering the solution to the oral cavity and/or throat structures (e.g., oropharynx, larynx, etc.). In some embodiments, the aqueous solution of HOCl may be diluted with one or more diluents. For example, approximately 0.5 ml to 20 ml of saline may be added to the aqueous solution of HOCl.

In some embodiments, illness suspected to be caused by adverse reactions to one or more medications and/or lifestyle choices may be treated by administering HOCl through the above described pulmonary delivery apparatuses. For example, oral inflammation and/or ulceration (e.g., mucositis) which may arise as an adverse effect to a particular medication (e.g., chemotherapy and radiotherapy treatment for cancer) or due to dehydration, poor mouth care, oxygen therapy, excessive use of alcohol and/or tobacco, and lack of protein in the diet may be treated by placing a particular amount of aqueous solution of HOCl (e.g., approximately 5 mL) may administered by the above described pulmonary delivery apparatuses. In some embodiments, the aqueous solution of HOCl may be diluted with one or more diluents. For example, approximately 0.5 ml to 20 ml of saline may be added to the aqueous solution of HOCl.

In some embodiments, illness suspected to be caused by microbes, bacteria, spores, viral, fungal, and/or allergy-causing agents may be treated by administering HOCl through administering the above described pulmonary delivery apparatuses.

In some embodiments, one or more effects of relaxing one or more respiratory structures (e.g., uvula, soft palate, etc.) resulting in a sound (e.g., snoring) due to their vibrations during sleep may be treated by administering HOCl. In some embodiments, the HOCl solution may be administered by a pulmonary delivery apparatuses, as described above.

In some embodiments, an irritation, inflammation, and/or obstruction of the breathing passages resulting in a cough reflex and often associated with acute and/or chronic respiratory tract infection may be treated by administering HOCl. In some embodiments, the HOCl solution may be administered by a pulmonary delivery apparatuses, as described above.

In some embodiments, an irritation and/or an inflammation of the voice box resulting in loss of voice and/or diminished capacity to produce sound (e.g., laryngitis) may be treated by administering HOCl. In some embodiments, the HOCl solution may be administered by a pulmonary delivery apparatuses, as described above.

In some embodiments, an irritation and/or an inflammation of one or more structures within the nasal cavity and/or throat due to an allergic reaction to one or more allergens, such as pet dander, dust, mites, pollen and mold, may be treated by administering HOCl. In some embodiments, the HOCl solution may be administered by a pulmonary delivery apparatuses, as described above. Alternatively, the HOCl solution may be used to decrease the histamine response which may be elevated during an allergic response to one or more allergens, as previously alluded. For example, the HOCl may be administered through the use of a HOCl containing oral rinse solution, a gargling solution, as described above, or a pulmonary delivery apparatuses.

In some embodiments, infectious disease agents, infectious diseases, and/or complications after infections (e.g., diseases caused by microbes (including spores), antimicrobes, pollutants, microorganisms, biofilms, viruses, bacteria, fungi, protists, parasites, allergy-causing agents, and/or other organisms, including mast cell degranulation, acne, pneumonia, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, Staphylococcus aureus, Haemophilius influenzae, klebsiella pnuemoniae, Pseudomona aeruginosa, Bordetella pertussis, Moraxella catarrhalis, Coxiella burnetiid, Chlamdyophilia pneumoniae, Mycoplasma pneumoniae, Legionella pneumophilia, Yesinia pestis, influenza viruses, rhinoviruses, respiratory syncytial virus, adenovirus, enterovirus, parainfluenza, Epstein-Barr virus, cytomegalovirus, hantavirus, Herpes simplex, Histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, and the like) may be treated with administration of HOCl via pulmonary administration.

Additionally, administration of HOCl via pulmonary delivery apparatuses may also prevent the spreading of microbials on medical equipment (e.g., medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, CPAP equipment, BiPAP equipment, asthma nebulizer, oxygen concentrators, and/or other medical equipment) used by patients and may further disinfect and/or clean the medical equipment through use. By administering HOCl to the medical equipment through the use of various delivery mechanisms, such as placing the HOCL in the reservoir of a nebulizer and then running the machine, the administration of HOCl can be bifunctional such that it treats the patient and cleans the medical equipment. The treatment may be administered, or delivered, for 5 seconds, 15 seconds, 30 seconds, 1 minute, 1 hour, 6 hours, 12 hours, etc. or any time period in between. The treatment may be administered 5 to 10 times a day for between 1 and 4 weeks. These ranges are merely exemplary and one skilled in the art would appreciate that other ranges are possible. It should be appreciated that in some embodiments the amount of solution administered, or delivered, is limited by the delivery mechanism (e.g., a spray into a nasal cavity is limited by the spray bottle and related components).

The delivery mechanisms may include bathing the pulmonary delivery apparatuses in an aqueous solution with HOCl, rinsing the pulmonary delivery apparatuses in an aqueous solution with HOCl, spraying the pulmonary delivery apparatus in an aqueous solution with HOCl, misting the pulmonary delivery apparatuses with an aqueous solution with HOCl, fogging the pulmonary delivery apparatuses in an aqueous solution with HOCl, aerosolizing the pulmonary delivery apparatuses with an aqueous solution with HOCl, electrostatically spraying the pulmonary delivery apparatuses with an aqueous solution with HOCl, placing HOCL within the pulmonary delivery apparatuses and operating the equipment to disseminate the aqueous solution with HOCL, and/or other delivery mechanisms.

For example, spraying the pulmonary delivery apparatuses with an aqueous solution with HOCl may include pumping a trigger on a container that lowers the air pressure within the tube that is in the bottle. The pumping action forces the solution up the tube into a smaller nozzle that is converted into a spray or a mist.

Similarly, misting the pulmonary delivery apparatuses with an aqueous solution with HOCl may include using a pressure pump to send a solution through a nozzle to turn the solution in a mist. Further, fogging the pulmonary delivery apparatuses with an aqueous solution with HOCl may include thermal foggers, ULV, or cold, foggers, and/or other foggers. Thermal foggers may use heat to vaporize a solution to be sprayed as a fog. The resulting particle size may be between about 0.5 to 10 microns. ULV foggers may use motors that produce a high power, low pressure air stream. The solution may be sent through the air stream through a nozzle that gives the resulting air flow a swirling motion separating it into tiny particles. The resulting particle size may be between about 5 to 30 microns.

Aerosolizing the pulmonary delivery apparatuses in an aqueous solution with HOCl may include pressurizing the solution within a container and using a propellant to push the solution into the air in aerosolized form and then what, are you spraying the pressurized solution onto the equipment, if so, then state that. For example, aerosolizing pulmonary delivery apparatuses (e.g., CPAP device, nebulizers, tubes, and mask) with an aqueous solution with HOCL may clean the pulmonary delivery apparatuses such that the pulmonary delivery apparatuses may not further spread microbials through extended use.

Additionally, operating pulmonary delivery apparatuses with an aqueous solution with HOCL such that the HOCL is distributed throughout the machine to disinfect and deodorize the pulmonary delivery apparatuses, provides a solution for cleaning the pulmonary delivery apparatuses and administering HOCl for the treatment of diseases such as tuberculosis when worn by the patient.

In some embodiments, infectious disease agents, infectious diseases, and/or complications after infections (e.g., diseases caused by microbes (including spores), antimicrobes, pollutants, microorganisms, biofilms, viruses, bacteria, fungi, protists, parasites, allergy-causing agents, and/or other organisms, including mast cell degranulation, acne, pneumonia, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, Staphylococcus aureus, Haemophilius influenzae, Klebsiella pnuemoniae, Pseudomona aeruginosa, Bordetella pertussis, Moraxella catarrhalis, Coxiella burnetiid, Chlamdyophilia pneumoniae, Mycoplasma pneumoniae, Legionella pneumophilia, Yesinia pestis, influenza viruses, rhinoviruses, respiratory syncytial virus, adenovirus, enterovirus, parainfluenza, tuberculosis, Epstein-Barr virus, cytomegalovirus, hantavirus, Herpes simplex, Histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, and the like) may prevent infection, therapeutically treat the infectious diseases, kill the pathogen causing the infectious disease, and/or limit progression of the infectious diseases by administering HOCl through use of various delivery mechanisms.

The pulmonary delivery apparatuses may include ingestion of an aqueous solution with HOCl to a patient via medical equipment, rinsing the affected area on and/or in the patient with the aqueous solution of HOCl (e.g., neti pot, liquid rinse, etc.), spraying the aqueous solution with HOCl onto or into the patient (e.g., nasal spray, throat spray, ear spray, etc.), misting the aqueous solution with HOCl onto or into the patient (e.g., nasal mist, throat mist, ear mist, etc.), fogging the aqueous solution with HOCl onto or into the patient, aerosolizing the aqueous solution with HOCl onto or into the patient, electrostatic, operating CPAP equipment with the aqueous solution with HOCL onto or into the patient, and/or other delivery mechanisms. It should be appreciated that the location where the HOCl solution is delivered may be different depending on the particular infectious disease. For example, treating an allergy-causing agent may be sprayed into a nasal cavity, while treating asthma may be sprayed into the mouth and/or throat. In another example, treating acne may be a liquid application to the affected area. As one example, tuberculosis infections in the lungs may be reactively treated, prevented, or prophylactically treated by delivering a HOCl solution via a nasal spray or rinse at about 180 PPM. Patients with MDR-TB may use HOCl to relieve and further treat the infection. HOCl may be used to prevent or prophylactically treat other infections. HOCl via a nasal spray or rinse at least once per day with a solution of about 180 PPM, may be used as a treatment to (i) prevent infection, (ii) kill the pathogen in the early stages shortly after exposure, and/or (iii) limit progression from the lungs. HOCl may be used to prophylactically treat an exposed or at-risk individual, thereby preventing infection, killing the pathogen early, and/or preventing migration from the lung.

In some embodiments, infections of the lungs and/or pneumonia or tuberculosis may be prevented or prophylactically treated by using a pulmonary delivery apparatuses containing an aqueous solution of HOCl at least once per day at 180 PPM of HOCl. Prophylaxis using HOCl may benefit following groups at risk for recurrent lung infections:

Immunosuppressed (e.g., due to hereditary immunodeficiency, HIV, chemotherapy, biologic therapy (e.g. anti-TNF), transplant, etc.)—These patients may be at a higher risk for pneumonia due to organisms that do not cause pneumonia in immunologically intact patients, as well as routine respiratory pathogens.

Asplenectics—Patients who have had their spleens removed may be susceptible to lung infections by pathogens, such as Streptococcus pneumoniae.

Cirrhosis—Patients with cirrhosis may be susceptible to lung infections by pathogens, such as S. pneumoniae.

Chronic lung diseases (e.g., cystic fibrosis, asthma, lung cancer, chronic obstructive pulmonary disease [COPD], chronic interstitial lung diseases, chronic pulmonary fibrosis, etc.)—Patients with chronic lung diseases may be at risk for pneumonia.

Autoimmune diseases—Autoimmune diseases (e.g., granulomatosis with granulomatous polyangiitis (GPA, formerly Wegner's Granulomatosis)) may affect the lungs and render it susceptible to infection. In these situations, the HOCl solution may be used to provide anti-inflammatory effects.

Contamination of respiratory assist devices—Tracheostomies, noninvasive positive pressure ventilation devices, ventilators, CPAP equipment, BiPAP equipment, asthma nebulizer, oxygen concentrators, etc. may become colonized with organisms (e.g., some that form biofilms within the tubing), and these organisms may infect the lungs and lead to pneumonia. As one example of minimizing the likelihood of organism colonization of the medical equipment, nebulized HOCl (e.g., about 100 ppm) may be applied directly into a respiratory assistance devices, thereby preventing the development of nosocomial pneumonia in patients using the respiratory assistance devices. As another example of minimizing the likelihood of organism colonization of medical equipment, HOCL may be used in place of distilled water in CPAP equipment, BiPAP equipment, asthma nebulizer, and oxygen concentrators to deodorize the equipment and disinfect both the equipment and patient's respiratory system. In some embodiments, HOCl can also be used to clean the masks and/or nasal pieces of the respiratory assist devices, such as CPAP equipment, BiPAP equipment, asthma nebulizer, and oxygen concentrators, as well as devices which can be used to treat pulmonary infections.

Tuberculosis—Patients with TB or MDR-TB.

Pneumonia—Patients with pneumonia or drug resistant pneumonia.

As another example, infections of the lungs and bronchi may be treated by pulmonary delivery apparatuses containing an aqueous solution of HOCl five to ten times per day. In some embodiments, HOCl may assist antibiotic therapy in clearing or decreasing the organism load from the lung cavity.

As another example, HOCl may be used to clean pulmonary delivery apparatuses that may be contaminated by organisms, some of which form biofilms. Bacterial biofilms are a source of infection when used for diagnostics and/or treatment. HOCl may be used to penetrate and kill bacteria within biofilms. HOCl may be in a misted or soaked medium to decontaminate the pulmonary delivery apparatuses (e.g., CPAP devices, nebulizers, and the like). Misted HOCl may also be used to decontaminate reused masks.

In some embodiments, the pH level of the HOCl solution administered through the methods disclosed herein may be pH-neutral because stabilized and/or pH-neutral HOCl is superior in terms of antimicrobial activity to non-stabilized HOCl and acidified bleach, including against hypochlorite-resistant strains. In some embodiments, the acidotic pH level of the HOCl may be within the range resulting in the highest amount of undissolved HOCl. For example, the acidotic pH level may range from approximately pH 5.1 to approximately pH 5.9. In another example, the acidotic pH level may range from approximately pH 4.0 to approximately pH 7.0.

In some embodiments, HOCl solution may be placed in the reservoir of a pulmonary delivery apparatuses to disinfect and deodorize the pulmonary delivery apparatuses, tubing, and mask, collectively the pulmonary delivery apparatuses. For example, the HOCl solution may be placed in the reservoir of the pulmonary delivery apparatuses and the pulmonary delivery apparatuses can be run without the patient wearing the mask for any amount of time ranging from one to thirty minutes. In another example, HOCl solution may be placed in the reservoir of the CPAP device and the CPAP device can be run with the patient wearing the mask for any amount of time ranging from one to thirty minutes.

In some embodiments, a mist comprising an aqueous HOCl solution may include one or more of the following: placing an aqueous solution of HOCl ranging from 0.1 ml to 10 ml into a spray bottle adequately suited for disinfecting an deodorizing, and administering the mist comprising aqueous solution HOCl into the mask, tubing, or pulmonary delivery apparatuses of the pulmonary delivery apparatuses by compressing the spray bottle. For example, approximately one to ten sprays may be administered into the mask for pulmonary delivery apparatuses by compressing the spray bottle. In another example, approximately one to ten sprays may be administered into or upon the tubing of the pulmonary delivery apparatuses by compressing the spray bottle. In another example, approximately one to ten sprays may be administered into or upon the pulmonary delivery apparatuses and pulmonary delivery apparatuses reservoir by compressing the spray bottle. In some embodiments, the aqueous solution of HOCl may be diluted with one or more diluents. For example, approximately 10 ml of saline may be added to 10 mL of aqueous solution of HOCl.

In some embodiments, the aforementioned method for cleaning pulmonary delivery apparatuses equipment can be used on CPAP or BiPAP equipment, asthma nebulizers, and oxygen concentrators, and/or such similar pulmonary devices.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical, or physical partitioning and configurations can be implemented to implement the desired features of the present disclosure. Also, a multitude of different constituent module names other than those depicted herein can be applied to the various partitions. Additionally, with regard to diagrams, operational descriptions, and method claims, the order in which the steps are presented herein shall not mandate that various embodiments be implemented to perform the recited functionality in the same order unless the context dictates otherwise.

Although the disclosure is described above in terms of various embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “typical,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “element” does not imply that the components or functionality described or claimed as part of the element are all configured in a common package. Indeed, any or all of the various components of an element can be combined in a single package or separately maintained and can further be distributed in multiple groupings or packages or across multiple locations.

Additionally, the various embodiments set forth herein are described in terms of exemplary diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method of treating drug resistant pneumonia or tuberculosis with hypochlorous acid, the method comprising:
delivering a solution to a patient via a pulmonary delivery apparatus, wherein the solution comprises an aqueous solution of hypochlorous acid, and wherein the hypochlorous acid is approximately 180 parts per million (ppm) of the aqueous solution.

2. The method of claim 1, wherein the hypochlorous acid has a pH of less than 6.

3. The method of claim 1, wherein delivering the solution comprises:
placing the aqueous solution into a reservoir of the pulmonary delivery apparatus;
aerosolizing the aqueous solution into particles; and
distributing the aerosolized particles of the aqueous solution to a patient.

4. The method of claim 3, wherein the aerosolized particles of the aqueous solution are between 0.1 μm and 99 μm in size.

5. The method of claim 1, wherein delivering the solution comprises:
placing the aqueous solution of the hypochlorous acid within a reservoir of a spray bottle;
transforming the aqueous solution of the hypochlorous acid into a mist; and
spraying the mist of the aqueous solution of hypochlorous acid into the nose or mouth of the patient.

6. The method of claim 1, wherein delivering the solution comprises:
placing the aqueous solution of the hypochlorous acid within a reservoir of an atomizer;
transforming the aqueous solution of the hypochlorous acid into a fine spray; and
distributing the fine spray of the aqueous solution of hypochlorous acid into the nose or mouth of the patient.

7. The method of claim 1, wherein the pulmonary delivery apparatus is selected from the group consisting of: CPAP devices, BiPAP devices, nebulizers, asthma nebulizers, oxygen concentrators, medical tubing, PAP tubing, and masks.

8. A method of prophylactically treating pneumonia or tuberculosis, the method comprising:
applying a solution to medical equipment used by a patient, thereby preventing infection by Mycobacterium tuberculosis bacteria on the medical equipment;
wherein the solution comprises an aqueous solution of hypochlorous acid, and wherein the hypochlorous acid is approximately 180 ppm of the aqueous solution.

9. The method of claim 8, wherein the hypochlorous acid has a pH of less than 6.

10. The method of claim 8, wherein applying the solution comprises:
placing the aqueous solution of the hypochlorous acid within a reservoir of an atomizer;
transforming the aqueous solution of the hypochlorous acid into a fine spray; and
distributing the fine spray of the aqueous solution of the hypochlorous acid to the medical equipment.

11. The method of claim 10, wherein particles in the solution are between 0.1 μm and 99 μm in size.

12. The method of claim 8, wherein the delivery of solution comprises:
placing the aqueous solution of the hypochlorous acid into a reservoir of a nebulizer;
aerosolizing the aqueous solution of hypochlorous acid into particles; and
distributing the aerosolized particles of the aqueous solution of hypochlorous acid onto an affected area of the patient.

13. The method of claim 8, wherein the medical equipment comprises CPAP equipment, BiPAP equipment, asthma nebulizers, oxygen concentrators, medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, asthma nebulizer, or oxygen concentrators.
